(19) United States
(12) Patent Application Publication
(10) Pub. No.: US 2006/0073481 A1
(43) Pub. Date: Apr. 6, 2006

(54) Formation and use of site specific nucleic acid coupled binding polypeptides

(75) Inventors: Ian E. Burbulis, Berkeley, CA (US); Robert H. Carlson, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Molecular Sciences Institute, Berkeley, CA (US)

(21) Appl. No.: 10/515,108

(22) PCT Filed: Apr. 23, 2003
(86) PCT No.: PCT/US03/12797

§ 371 Date: Nov. 19, 2004

Related U.S. Application Data
(60) Provisional application No. 60/374,795, filed on Apr. 23, 2002.

(51) Int. Cl. C12Q 1/68; C12N 15/87

(52) U.S. Cl. 435/6; 435/455

(57) ABSTRACT

The present invention provides methods of covalently attaching a nucleic acid to a specific site on a binding polypeptide non-enzymatically. The methods produce large quantities of site specific nucleic acid coupled binding polypeptides. The site specific nucleic acid coupled binding polypeptides are used in accurate and sensitive methods for detecting and quantifying target analytes. Thus, the present invention provides economical and facile methods of making site specific nucleic acid coupled binding polypeptides for use in detection of target analytes.

### CROSS-REFERENCES TO RELATED APPLICATIONS

This application claims priority to U.S. provisional patent application No. 60/374,795 filed Apr. 23, 2002 and U.S. patent application Ser. No. 10/218,233, filed Aug. 12, 2002, which are incorporated herein by reference in their entirety for all purposes and are all assigned to the same assignee as the present application.

### BACKGROUND OF THE INVENTION

Detection and quantification of chemical and biological molecules is of central importance in both academic and industrial areas. A variety of detection methods are currently available, including ELISA based immunoabsorbent assays, protein biochips, and the like. Recently, however, more sensitive detection methods have developed based on the use of nucleic acid tagged polypeptide molecules.

The detection of analytes using nucleic acid tagged polypeptide molecules provides sensitive detection with rapid response time. Typically, the nucleic acid tagged polypeptide comprises a polypeptide portion that binds the analyte and a nucleic acid tag that provides a detectable signal. The nucleic acid tag is usually detected by amplification through polymerase chain reaction techniques (PCR). The resulting nucleic acid amplification products are then detected by established methods such as gel electrophoresis or high performance liquid chromatography (HPLC). The detection of the nucleic acid amplification products correspond to the presence of the analyte bound by the polypeptide portion of the nucleic acid tagged polypeptide.

Currently available nucleic acid tagged polypeptide molecules have several disadvantages. For example, nucleic acids may be non-covalently bound to the polypeptide portion of the nucleic acid tagged polypeptide (e.g. the biotin-streptavidin linking system). However, this non-covalent interaction is easily disrupted under a variety of solution conditions. Therefore, non-covalently nucleic acid tagged polypeptides are of limited utility. In other methods, nucleic acids are non-specifically covalently bound to the polypeptide portions using known crosslinkers such as glutaraldehyde. However, these methods produce nucleic acid tagged polypeptides containing an undefined number of nucleic acid tags at unknown locations on the polypeptide. Thus, detection and quantitation results using crosslinked nucleic acid tagged polypeptides are ambiguous or inaccurate. Another method of attaching a nucleic acid to a polypeptide involves the use of a ribosomal enzyme to covalently link a puromycin substituted nucleic acid to a polypeptide. However, these enzymatic methods are low yielding, time consuming and expensive.

Thus, there is a need in the art for sensitive and efficient methods of detecting and quantifying chemical and biological molecules. In addition, there is a need in the art for a method of site specifically attaching a nucleic acid to a polypeptide that is both inexpensive and efficient. The current invention solves these and other problems.

### BRIEF SUMMARY OF THE INVENTION

The current invention provides methods of covalently attaching a nucleic acid to a specific site on a binding polypeptide without the use of expensive enzymes. The methods produce large quantities of site specific nucleic acid coupled binding polypeptides. The site specific nucleic acid coupled binding polypeptides are used in accurate and sensitive methods for detecting and quantifying target analytes. Thus, the present invention provides economical and facile methods of making site specific nucleic acid coupled binding polypeptides for use in detection of target analytes.

Thus, in a first aspect, the present invention provides a method of forming a site specific nucleic acid coupled binding polypeptide. The method includes introducing into a cell a recombinant DNA molecule comprising a nucleotide sequence encoding a recombinant binding polypeptide-intein molecule, wherein the recombinant binding polypeptide-intein molecule comprises an intein moiety genetically engineered into a predetermined site on a binding polypeptide moiety. The recombinant binding polypeptide-intein molecule is isolated from the cell. The isolated polypeptide-intein molecule is contacted with a nucleophile under conditions which permit substitution of the with the nucleophile to yield a binding polypeptide intermediate. The resulting binding polypeptide intermediate is contacted with a substituted nucleic acid under conditions which permit site specific bond formation between the substituted nucleic acid and the binding polypeptide to form a site specific nucleic acid coupled binding polypeptide.

In a second aspect, the present invention provides methods of detecting the presence of a target analyte in a sample. The methods include contacting a target analyte with a site specific nucleic acid coupled binding polypeptide to form a target analyte complex, wherein said site specific nucleic acid coupled binding polypeptide comprises a nucleic acid moiety and a binding polypeptide moiety to which the target analyte specifically binds. The nucleic acid moiety of said target analyte complex is contacted with a nucleic acid polymerase to form a plurality of nucleic acid detectors. The nucleic acid detectors are detected thereby detecting the presence of the target analyte in the sample.

### BRIEF DESCRIPTION OF THE DRAWINGS

FIG. 1 is a photograph of a gel illustrating an exemplary method of producing a recombinant binding polypeptide-intein molecule.

FIG. 2 is a photograph of a gel illustrating an exemplary method of forming a substituted double stranded nucleic acid.

FIG. 3A is a chromatogram illustrating an exemplary method of partially purifying a site specific nucleic acid coupled binding polypeptide.

FIG. 3B is a chromatogram illustrating an exemplary method of purifying a site specific nucleic acid coupled binding polypeptide.

FIG. 3C is a photograph of a gel illustrating an exemplary method of purifying a substituted double stranded nucleic acid.

FIG. 4 is a digital representation of a gel illustrating an exemplary method of contacting a target analyte with a site specific substituted nucleic acid coupled polypeptide to form a target analyte complex.

### DETAILED DESCRIPTION OF THE INVENTION

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, genetic engineering, recombinant DNA technology, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry and organic synthetic chemistry described below are those well known and commonly employed in the art. Standard techniques, or modifications thereof, are used for chemical syntheses and chemical analyses.

As used herein, “nucleic acid” means DNA, RNA, single-stranded, double-stranded, or more highly aggregated hybridization motifs, and any chemical modifications thereof Modifications include, but are not limited to, those providing chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore or another moiety.

A “recombinant” molecule, as used herein, refers to molecules derived from or containing genes or parts of genes, in which the genes or parts of genes are combined using genetic engineering techniques. Thus, a “recombinant DNA molecule” is a DNA molecule comprising genes or parts of genes combined by genetic engineering techniques and a “recombinant binding polypeptide-intein molecule” is a molecule comprising a binding polypeptide and an intein combined using genetic engineering techniques.

A “predetermined site,” as used herein, refers to a specific site on a first molecule to which a second molecule is intended to be attached thereto.

“Moiety” refers to a component, part or portion of a chemical molecule. For example, a “nucleic acid moiety” refers to the portion of a molecule containing a nucleic acid and a “binding polypeptide moiety” refers to the portion of a molecule containing a binding polypeptide.

“Site specific nucleic acid coupled binding polypeptide” refers to a nucleic acid moiety that is covalently bound to a binding polypeptide at a specific location on or within the binding polypeptide. Typically the covalent bond is an amide bond, an ester bond, or a thioester bond.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “eptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid,” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g. hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

“Binding polypeptide” refers to a peptide capable of specifically binding to a target analyte. “Specifically binding” or “specifically binds” refers to the strength of the binding interaction between two molecules. Typically, two molecules that specifically bind will have a dissociation constant (K_{D}) in at least the micromolar range. Binding peptides may bind through a variety of known intermolecular interactions such as hydrogen bonding, van der Waals forces, and hydrophobic interactions. “Coupled,” as used herein, refers to an attachment via a covalent bond.

“Intein,” as used herein, refers to a peptide or peptide moiety that is capable of being displaced (also referred to as excised or cleaved) by a nucleophile when the intein is covalently bound to a binding polypeptide. An intein has at least one cysteine, serine, or threonine amino acid residue capable of forming a thioester bond (in the case of cysteine) or ester bond (in the case of serine or threonine) between the binding polypeptide moiety and the intein moiety of a recombinant binding polypeptide-intein molecule. The cysteine sulfhydryl or the serine or threonine hydroxyl act as nucleophiles to displace the amino functionality of an amide bond that links the intein moiety to the binding polypeptide moiety.

“Nucleophile”, as used herein, refers to a molecule having a nucleophilic atom with a nonbonded pair of electrons capable of forming a covalent bond with an electron pair acceptor. Examples of nucleophilic atoms include, for example, the oxygen of a hydroxyl moiety, the nitrogen of an amino moiety, or the sulfur of a sulfhydryl moiety. A variety of electron pair acceptors (also referred to in the art as Lewis acids) are useful in the current invention, including the carbon atom of a carboxyl moiety. Where the nucleophile is covalently bonded to another molecule, it is referred to as a “nucleophile moiety.” For example, thiophenol is a nucleophile containing the nucleophilic sulfur atom. When the thiophenol is bonded to a binding polypeptide through a covalent thioester linkage, the thiophenol nucleophile is a “nucleophile moiety.”

“Thioester” refers to a heteroalkylene (as defined below) having a —C(O)S— moiety. Thus, a “thioester bond” refers to a covalent bond having a thioester heteroalkylene.

“Binding polypeptide intermediate” refers to a binding polypeptide covalently bound to a nucleophile moiety. A “binding polypeptide-thioester intermediate” refers to a binding polypeptide intermediate wherein a binding polypeptide covalently bound to a nucleophile moiety via a thioester bond. A “binding polypeptide-ester intermediate” refers to a binding polypeptide intermediate wherein a binding polypeptide covalently bound to a nucleophile moiety via an ester bond.

A “substituted nucleic acid,” as used herein, refers to a nucleic acid substituted with at least one nucleophilic group. A “nucleophilic group,” as used herein, refers to a moiety such as a sulfhydryl, amino or hydroxyl, that is capable of forming a covalent bond with an electron pair acceptor. A “sulfhydryl-substituted nucleic acid” refers to a substituted nucleic acid which is substituted with at least one sulfhydryl nucleophilic group. A “(bis)substituted nucleic acid” refers to a double stranded nucleic acid substituted with at least one nucleophilic group at each end. A “sulfhydryl” refers to —SH, a “hydroxyl” refers to —OH and an “amino” refers to —NH₂.

“Target analyte” refers to a molecule for which detection is desired.

“Non-enzymatically” refers to the absence of an enzyme or enzymes in a given process or method. An “enzyme” refers to a protein that catalyzes a specific reaction wherein the enzyme is not consumed in the reaction. Thus, an “enzyme” is able to catalyze multiple reactions at a defined turnover rate.

The term “isolated” refers to a material that is substantially or essentially free from components, which are used to produce the material. For example, a recombinant binding polypeptide-intein molecules of the invention is “isolated” when it is substantially or essentially free from components that normally accompany the material in the mixture used to prepare the recombinant binding polypeptide-intein molecules. “Isolated” and “pure” are used interchangeably. Typically, molecules of the invention have a level of purity preferably expressed as a range. The lower end of the range of purity for the molecules is about 60%, about 70% or about 80% and the upper end of the range of purity is about 70%, about 80%, about 90% or more than about 90%.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (ie. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropyhnethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, and —CH═CH—(CH₃)—CH₃. Up to two heteroatoms may be consecutive, such as, for example, —CH₂—NH—OCH₃ and —CH₂—O—Si(C—H₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. In addition, the term “cycloalkylene” by itself or as part of another substituent means a divalent radical derived from a cycloalkyl includes those groups described as “heterocycloalkylene,” meaning the divalent radical derived from a heterocycloalkyl.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. In addition, the term “arylene” by itself or as part of another substituent means a divalent radical derived from an aryl and includes those groups described as “heteroaryl,” meaning the divalent radical derived from a heteroaryl.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)—NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, alkyl, heteroalkyl, aryl and heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula —T—C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O— , —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

Introduction

The present invention provides methods of preparing site specific nucleic acid coupled binding polypeptides. The methods disclosed herein represent a significant advance in the art of producing site specific nucleic acid coupled binding polypeptides. In contrast to previously known methods, the current invention allows site specific nucleic acid coupled binding polypeptides to be produced cheaply and in high quantities. The invention also provides methods of detecting a target analyte using nucleic acid coupled binding polypeptides.

### I. Forming A Site Specific Nucleic Acid Coupled Binding Polypeptide

In a first aspect, the present invention provides methods of forming a site specific nucleic acid coupled binding polypeptide. The nucleic acid coupled binding polypeptides are formed by covalently binding a nucleic acid to a specific site on a binding polypeptide.

In one embodiment, the covalent bond is formed non-enzymatically (defined above). The non-enzymatic method includes introducing into a cell a recombinant DNA molecule comprising a nucleotide sequence encoding a recombinant binding polypeptide-intein molecule, wherein the recombinant binding polypeptide-intein molecule comprises an intein moiety genetically engineered into a predetermined site on a binding polypeptide moiety. The binding polypeptide-intein molecule is isolated from the cell. The isolated polypeptide-intein molecule is contacted with a nucleophile under conditions which permit substitution of the intein moiety with the nucleophile to yield a binding polypeptide intermediate. The binding polypeptide intermediate contains a binding polypeptide moiety and a nucleophile moiety covalently linked through a first bond. The binding polypeptide intermediate is contacted with a nucleophile-substituted nucleic acid under conditions which permit substitution of the nucleophile moiety with the substituted nucleic acid to form a site specific nucleic acid coupled binding polypeptide. The site specific nucleic acid coupled binding polypeptide contains a binding polypeptide moiety and a substituted nucleic acid moiety covalently linked through a second bond.

In one embodiment, the first bond and second bonds are thioester bonds and the substituted nucleic acid is a sulfhydryl-substituted nucleic acid. In another embodiment, the substituted nucleic acid further comprises a second nucleophilic group The second nucleophilic group displaces the first nucleophilic group to form a third bond between the binding polypeptide moiety and the substituted nucleic acid moiety. In an exemplary embodiment, the third bond is an amide and the second nucleophilic group is an amino.

A variety of methods are described below (see sections A-C below) for non-enzymatically forming the covalent bond between the nucleic acid and the binding polypeptide. These methods include the formation of a recombinant binding polypeptide-intein molecule. Non-enzymatic methods of forming the covalent bond between the nucleic acid and the binding polypeptide provide several advantages over known enzymatic methods, such as those of Szostak et al., WO 98/31700; Roberts et al., Proc. Natl. Acad. Sci. 94: 12297-12302 (1997); Szostak et al., U.S. Pat. Nos. 6.258,558 B1 and U.S. Pat. No. 6,261,804 B1, which are herein incorporated by reference for all purposes. For example, the non-enzymatic methods using recombinant binding polypeptide-intein molecules presented herein provide higher yields, are more efficient, and are significantly cheaper than the known enzymatic methods.

In an exemplary embodiment, an intein is genetically engineered into a non-native and predetermined site on a binding polypeptide to produce a recombinant binding polypeptide-intein molecule. The intein moiety contains a reactive amino acid, typically a reactive cysteine, threonine or serine. For clarity of illustration, the present exemplary embodiment is illustrated with a reactive cysteine. The reactive cysteine of the intein moiety attacks the amide linkage between the intein and binding polypeptide resulting in an N to S acyl shift. The recombinant binding polypeptide-intein molecule is contacted with a nucleophile under conditions which permit the substitution of the intein moiety with the nucleophile to yield a binding polypeptide intermediate. For clarity of illustration, the present exemplary embodiment is illustrated with a thiophenol nucleophile resulting in a binding polypeptide thioester intermediate containing a thioester first bond. The intermediate binding polypeptide thioester intermediate is then contacted with a substituted nucleic acid under conditions which permit substitution of the thiophenol nucleophile moiety with the substituted nucleic acid to form a site specific nucleic acid coupled binding polypeptide containing a second bond. For clarity of illustration, the present exemplary embodiment is illustrated with a sulfhydryl-substituted nucleic acid moiety resulting in a site specific nucleic acid coupled binding polypeptide containing a thioester second bond.

In other exemplary embodiment, a second nucleophilic group on the sulfhydryl-substituted nucleic acid moiety attacks the thioester second bond resulting in an a third bond. In an exemplary embodiment, the second nucleophilic group is an amino nucleophilic group that forms an amide third bond.

In another exemplary embodiment, an antibody-intein molecule is isolated and reacted with a thiophenol nucleophile under conditions which permit the substitution of the intein moiety with the thiophenol to yield an antibody-thioester intermediate. The resulting antibody-thioester intermediate comprises an antibody moiety covalently linked to the thiophenol though a first thioester bond. The first thioester bond of the antibody-thioester intermediate is then contacted with a sulfhydryl-substituted nucleic acid under conditions which permit substitution of the nucleophile moiety with the sulfhydryl-substituted nucleic acid to form a site specific nucleic acid coupled binding polypeptide comprising a binding polypeptide moiety and a sulfhydryl-substituted nucleic acid moiety covalently linked through a second thioester bond.

The mechanisms of natural intein mediated protein splicing are well characterized. See Noren et al, Angewandte Chemie Int. Ed. 39:450-466 (2000). Inteins useful in the current invention include only those in which the excision or cleavage of the intein moiety from a peptide moiety N-terminal to the intein moiety (hereinafter referred to as the “N-terminal peptide moiety”) can be controlled by the addition of a nucleophile. The inteins of the present invention comprise at least one cysteine, serine, or threonine amino acid residue capable of forming a thioester bond (in the case of cysteine) or ester bond (in the case of serine or threonine) between the N-terminal peptide moiety and the intein moiety of a recombinant binding peptide-intein molecule. The cysteine sulfhydryl or the serine or threonine hydroxyl displaces the amino functionality of an amide bond that links the intein moiety to the N-terminal peptide moiety. The resulting ester or thioester bond is susceptible to a nucleophilic substitution from a variety of nucleophiles resulting in displacement of the intein moiety from the N-terminal peptide.

In another embodiment, the covalent bond between the nucleic acid and the binding polypeptide is formed enzymatically. In an exemplary embodiment, a ribosome catalyzes the covalent bond formation between a binding polypeptide and a nucleic acid molecule. Methods of using a ribosome to form site specific nucleic acid coupled binding polypeptides are discussed in detail in Szostak et al., WO 98/31700; Roberts et al., Proc. Natl. Acad. Sci. 94: 12297-12302 (1997); Szostak et al., U.S. Pat. Nos. 6,258,558 B1 and U.S. Pat. No. 6,261,804 B1, which are herein incorporated by reference for all purposes.

A. Introducing into a Cell a Recombinant DNA Encoding a Binding Polypeptide-Intein Molecule

In another embodiment, the present invention provides methods of producing a site specific nucleic acid coupled binding polypeptide by non-enzymatically forming a covalent bond between a nucleic acid and a specific site on a binding polypeptide. The first step typically involves introducing into a cell a recombinant DNA molecule comprising a nucleotide sequence encoding a recombinant binding polypeptide-intein molecule, wherein the recombinant binding polypeptide-intein molecule comprises an intein moiety genetically engineered into a predetermined site on a binding polypeptide moiety. The recombinant DNA molecule is typically produced by genetically engineering an intein into a predetermined site within, adjacent to, or near a DNA sequence encoding a binding polypeptide to produce a recombinant binding polypeptide-intein molecule.

The use of a recombinant binding polypeptide-intein molecule in forming the site specific nucleic acid coupled binding polypeptides has several advantages. First, by recombinantly producing binding polypeptide-intein molecules, a higher quantity is produced than with peptide synthesis techniques. A higher quantity of binding polypeptide-intein molecules allows for higher production of site specific nucleic acid coupled binding polypeptides. In addition, current peptide synthesis techniques produce polypeptides of limited size whereas recombinant techniques have been used to produce polypeptides of relatively large size. Furthermore, current methods of folding polypeptides in vitro to form functional proteins are highly unpredictable, costly and inefficient. For these and other reasons, the use of recombinant binding polypeptide-intein molecules represents a significant step forward in the art of coupling nucleic acids to polypeptides.

A variety of genetic engineering and recombinant DNA techniques are useful in the current invention. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference for all purposes). More specific methods of engineering an intein onto a specific site of a polypeptide are discussed in detail, for example, in Comb et al., U.S. Pat. No. 5,834,247, which is herein incorporated by reference for all purposes. In addition, a variety of intein based cloning vectors are commercially available from New England Biolabs.

In an exemplary embodiment, the DNA encoding an intein is inserted into an appropriate expression vector (i.e., a vector which contains the necessary elements for the transcription and translation of the inserted binding polypeptide-intein sequence). A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors; bacteria transformed with bacteriophage DNA; plasmid DNA; and cosmid DNA. Depending on the host-vector system utilized, any one of a number of suitable transcription and translation elements may be used. For example, when expressing a modified eukaryotic protein, it may be advantageous to use appropriate eukaryotic vectors and host cells. Expression of the fusion DNA results in the production of the modified proteins of the present invention.

In another exemplary embodiment, the DNA encoding an intein is inserted into an expression vector at a restriction enzyme site that makes a blunt cut in the 3′ end of the DNA encoding the binding polypeptide and which is in frame. In another exemplary embodiment, the intein DNA fragment is synthesized with a threonine codon, serine codon, or cysteine codon at its 5′ end. This fragment is then ligated in-frame to a linear plasmid and cut to blunt ends by the restriction endonuclease.

In another exemplary embodiment, a linear form of the plasmid is generated using PCR then the linear plasmid is ligated to the intein DNA fragment. Typically, the plasmid vector carrying the binding polypeptide sequence is relatively small, for example, less than about 5 Kb. Using this method the intein gene can be inserted at any location in the polypeptide gene.

Binding polypeptides of the current invention include any appropriate polypeptide capable of binding a target molecule. Target molecules include, for example, proteins, carbohydrates, a nucleic acids, lipids, vitamins, viruses, bacteria, metals. Binding polypeptides may belong to a variety of binding polypeptide classes such as antibodies, hormones, receptors, protein binding domains, and portions thereof. Exemplary binding proteins include maltose or arabinose binding protein, cell-surface receptors (e.g. platelet derived growth factor receptor and epidermal growth factor receptor), streptavidin, chitin binding protein, antibodies (e.g. monoclonal antibodies, polyclonal antibodies, antibody fragments such as single-chain fragment variable regions, and antibody derivatives), carbohydrate-binding lectins (of plant and animal origin), antibody fragments, single-chain variable fragments, and protein motif binding polypeptides (e.g. SH₂ domain).

Once obtained, the recombinant binding polypeptide-intein molecule can be separated and purified by appropriate known techniques or combinations thereof These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatography, methods utilizing a difference in hydrophobicity such as reverse-phase high performance liquid chromatography and methods utilizing a difference in isoelectric point, such as isoelectric focusing electrophoresis.

In an exemplary embodiment, the intein moiety contains a polypeptide purification tag that is genetically engineered into a specific site on the intein molecule. The specific site is typically chosen such that the polypeptide purification tag remains attached to the intein molecule upon excision. Polypeptide purification tags useful in the current invention include, for example, streptavidin, biotin, glutathione-S-transferase (GST), maltose-binding domain, chitinase (e.g. chitin binding domain), cellulase (cellulose binding domain), thioredoxin, protein G, protein A, Histidine-6, protein kinase inhibitor, c-Myc, and the like.

B. Contacting the Recombinant Binding Polypeptide-Intein Molecule with a Nucleophile

In another embodiment, the present invention provides methods of non-enzymatically producing a site specific nucleic acid coupled binding polypeptide that include contacting the recombinant binding polypeptide-intein molecule with a nucleophile to form a binding polypeptide intermediate.

Nucleophiles useful in the current invention are capable of displacing the intein moiety of the recombinant binding polypeptide-intein molecule to form a binding polypeptide intermediate. The binding polypeptide intermediate comprises a first bond between the binding polypeptide moiety and the nucleophile moiety. Typically, the first bond is an ester or thioester bond between the binding polypeptide moiety and the nucleophile moiety. Thus, the nucleophile is typically an alcohol or a thiol. In an exemplary embodiment, a binding polypeptide ester intermediate is formed using an alcohol nucleophile

In another exemplary embodiment, a binding polypeptide-thioester intermediate is formed using a thiol nucleophile. In another exemplary embodiment, the thiol nucleophile has the formula HS—R, wherein R is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In another exemplary embodiment, R is a substituted or unsubstituted heteroalkyl or a substituted or unsubstituted aryl. In another exemplary embodiment, R is thiophenol or 2-mercaptoethanesulfonic acid.

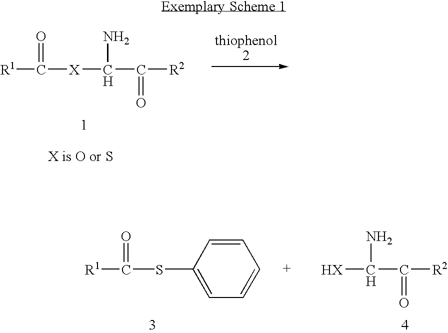

An exemplary scheme is presented above in Exemplary Scheme 1. A recombinant binding polypeptide-intein molecule 1 is contacted with the thiophenol nucleophile 2 to form the binding polypeptide-thioester intermediate 3, comprising a thioester first bond. The sulfhydryl group of 2 displaces the intein moiety of 1 resulting and formation of the displaced intein 4.

R′ contains a binding polypeptide moiety, or portion thereof, that is directly or indirectly covalently bound to X. Similarly, R² contains an intein moiety, or portion thereof, that is directly or indirectly covalently bound to X. Where R² or R³ is indirectly covalently bound to X, a peptidyl linker moiety connects R² or R³ to X. The peptidyl linker moiety is typically less than about 100 amino acids in length.

Typically, the recombinant binding polypeptide-intein molecule is contacted with a nucleophile under conditions which permit the substitution of the intein moiety with the nucleophile to yield a binding polypeptide-thioester intermediate. A variety of parameters may be considered in determining the appropriate reaction conditions that permit substitution of the intein moiety. Important parameters include, for example, temperature, ionic strength of the solution, pH, and the like. In an exemplary embodiment, reaction conditions for the substitution of the intein moiety are optimized by adjusting parameters such as those disclosed above.

In an exemplary embodiment, the natural amino acid intein sequence comprises an N-terminal serine or cysteine and a C-terminal serine, threonine or cysteine residue. To optimize the control of the intein excision, the C-terminal serine, threonine or cysteine is substituted with an alanine. This mutation allows the N-terminal serine or cysteine to form the desired thioester bond between the intein moiety and the binding polypeptide moiety while preventing the C-terminal serine, threonine or cysteine residue from prematurely excising the intein moiety.

A variety of inteins are useful in the present invention such as natural intein sequences, mutated natural intein sequences or completely non-natural intein sequences. Inteins may be any appropriate length. In an exemplary embodiment, the intein length is less than about 500 amino acids and at least about 1 amino acid. In another exemplary embodiment, the intein length is between at least about 100 amino acids and less than about 300 amino acids. The intein amino acid sequences are typically chosen such that the amino acid side chains of the intein do not interfere with the substitution of the intein moiety by the nucleophile. In an exemplary embodiment, the intein amino acid sequence of the intein is unaltered from its natural sequence. In another exemplary embodiment, the natural amino acid intein sequence has been mutated to optimize properties for the current invention. Many natural inteins contain C-terminal amino acids capable of displacing the intein from the binding polypeptide. See Noren et al., Angewandte Chemie Int. Ed. 39:450-466 (2000). For example, a nucleophilic C-terminal serine, threonine or cysteine the intein may disrupt the ester or thioester bond that links the binding polypeptide moiety to the intein moiety. Next, a C-terminal asparagine may excise the intein from the binding polypeptide completely. As a result, no binding-polypeptide intermediate is formed. To prevent this result, the natural intein sequence is mutated to delete or replace the C-terminal amino acids capable of displacing the intein from the binding polypeptide.

Thus, in another embodiment, the intein is a mutated form of a natural intein wherein the C-terminal amino acids capable of displacing the intein from the binding polypeptide are deleted or replaced. In an exemplary embodiment, the amino acids are replaced with an alanine. In another exemplary embodiment, the amino acids are deleted.

C. Contacting the Binding Polypeptide Intermediate with a Substituted Nucleic Acid

In another embodiment, the present invention provides methods of non-enzymatically producing a site specific nucleic acid coupled binding polypeptide that include contacting a binding polypeptide intermediate with a substituted nucleic acid. The substituted nucleic acid contains at least one first nucleophilic group. The resulting site specific nucleic acid coupled binding polypeptide contains a binding polypeptide moiety and a substituted nucleic acid moiety covalently linked through a second bond.

Substituted nucleic acids of the present invention contain at least one nucleophilic group (i.e. the first nucleophilic group) capable of displacing the nucleophilic moiety of the binding polypeptide intermediate to form a site specific nucleic acid coupled binding polypeptide. Useful first nucleophilic groups include, for example, sulfhydryl and hydroxyl groups. Thus, in an exemplary embodiment, the substituted nucleic acid is a sulfhydryl substituted nucleic acid and the second bond is a thioester. In another exemplary embodiment, the substituted nucleic acid contains is a hydroxyl substituted nucleic acid and the second bond is an ester.

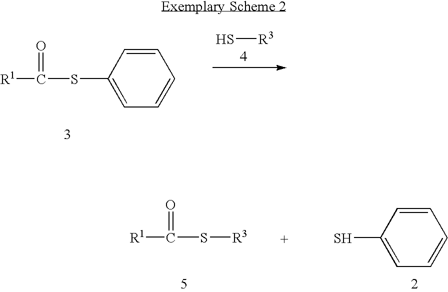

An exemplary scheme is presented above in Exemplary Scheme 2. The thiophenol nucleophile moiety of 3 is displaced by the sulfhydryl of the sulfhydryl-substituted nucleic acid 4. The nucleophilic displacement results in the formation of the site specific nucleic acid coupled binding polypeptide 5 and the displaced thiophenol nucleophile 2.

R³ contains a nucleic acid moiety that is directly or indirectly covalently bound to the sulfhydryl nucleophilic group. Where R³ is indirectly covalently bound to the sulfhydryl moiety, a linker moiety connects the nucleic acid moiety and the nucleophilic group. Any appropriate linker moiety may be used, including a substituted or unsubstituted alkylene or substituted or unsubstituted heteroalkylene.

Nucleic acid moieties of the site specific nucleic acid tagged binding polypeptides may be of any appropriate sequence or length. In an exemplary embodiment, the nucleic acid moiety is greater than about 10 nucleotides in length and less than about 500 nucleotides in length. In another exemplary embodiment, the nucleic acid moiety are greater than about 50 nucleotides in length and less than about 150 nucleotides in length.

In another embodiment, the substituted nucleic acid further contains a second nucleophilic group capable of displacing the first nucleophilic group. The second nucleophilic group is capable of attacking the second bond (typically an ester or thioester) between the first nucleophilic group and the binding polypeptide resulting in an acyl shift and the formation of a third bond. In an exemplary embodiment, the second nucleophilic group is an amino group capable of producing an O to N or S to N acyl shift. Thus, the resulting third bond is an amide bond. In another exemplary embodiment, the second nucleophilic group is a hydroxyl group capable of producing O to O or S to O acyl shift. Thus, the resulting third bond is an ester bond. Where the substituted nucleic acid contains a first nucleophilic group and a second nucleophilic group, it is referred to herein as a di-substituted nucleic acid.

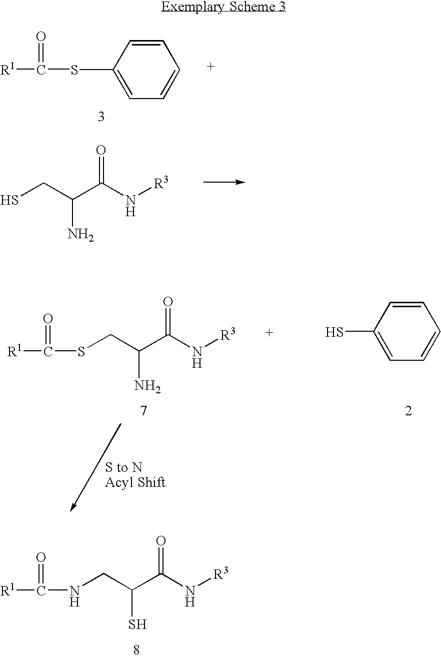

An exemplary scheme is presented above in Exemplary Scheme 3. The thiophenol nucleophile moiety of 3 is displaced by the sulfhydryl of the di-substituted nucleic acid 6. The nucleophilic displacement results in the formation of the site specific nucleic acid coupled binding polypeptide 7 comprising a thioester bond. The subsequent S to N acyl shift yields the site specific nucleic acid coupled binding polypeptide 8 containing an amide bond as the third bond.

A variety of methods are useful in forming the substituted nucleic acids of the present invention. In an exemplary embodiment, a substituted nucleic acid is formed by coupling a phosphoramidite containing a nucleophilic group to a solid phase or solution phase nucleic acid. See Beaucage et al., Tetrahedron Lett. 22: 1859 (1981), Eckstein et al., Oligonucleotides and Analogues: A Practical Approach, (1991), and Stetsenko et al., J. Org. Chem. 65: 4900-4908 (2000).

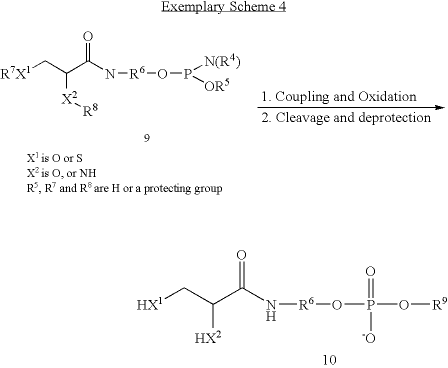

An exemplary solid phase synthesis method is illustrated above in Exemplary Scheme 4. The nucleophile-protected phosphoramidite 9 is first coupled to a solid support bound oligonucleotide using standard phosphoramidite coupling techniques. See Beaucage et al., Tetrahedron Lett. 22: 1859 (1981) and Stetsenko et al., J. Org. Chem. 65: 4900-4908 (2000). The support bound oligonucleotide is then oxidized and cleaved from the solid support. The symbols R⁵, R⁷, and R⁸ represent hydrogen or a protecting group. The symbol R⁹ represents a nucleic acid moiety.

The nucleophilic groups X¹ and X² may be deprotected before, during or after cleavage from the solid support to yield the di-substituted nucleic acid 10. In an exemplary embodiment, the second nucleophilic group is not deprotected until after the intein moiety is displaced.

The alkylene group R⁶ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In an exemplary embodiment, R⁶ is a substituted or unsubstituted cycloalkylene. In another exemplary embodiment, R⁶ is cyclohexylene.

The amino substituent R⁴ represents a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In an exemplary embodiment, R⁴ is a substituted or unsubstituted alkyl. In another exemplary embodiment, R⁴ is an unsubstituted 1-10 membered alkyl. In another exemplary embodiment, R⁴ is isopropyl or isobutyl. It should be understood that the two R⁴ groups attached to the single nitrogen are optionally different groups.

A variety of protecting groups may be used in Exemplary Scheme 4. A detailed description of protecting group strategies for hydroxyl, sulfhydryl and amino moieties are presented, for example, in Green et al., Protective Groups on Organic Synthesis, (1991); Stewart et al., Solid Phase Peptide Synthesis (1984); and Eckstein et al., Oligonucleotides and Analogues: A Practical Approach (1991). Thus, any appropriate protecting group may be used such as, for example, base labile protecting groups, acid labile protecting groups and protecting groups that are labile under oxidative or reductive conditions. In an exemplary embodiment, X¹ is a sulfhydryl nucleophilic group and R⁷ is S-tert-butyl sulfonyl. In another exemplary embodiment, X² is an amino protecting group and R⁸ is 9-fluorenyl-methyloxycarbonyl (Fmoc). In another exemplary embodiment, R⁸ is 2-cyanoethyl.

In another exemplary embodiment, a substituted nucleic acid is formed by coupling a nucleotide triphosphate containing a nucleophilic group to a nucleic acid. Typically, an enzyme is used to couple the triphosphate to a nucleic acids molecule. A variety of nucleotide triphosphates containing a nucleophilic group are useful in the current invention. In an exemplary embodiment, the nucleotide triphosphate containing a nucleophilic group is a substrate for a nucleic acid ligase or polymerase enzyme.

TABLE 1

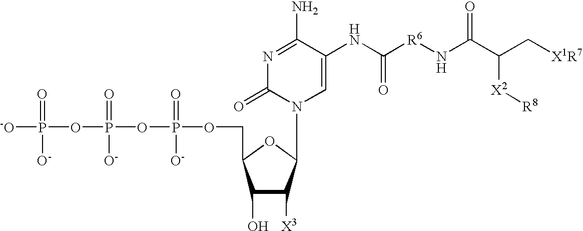

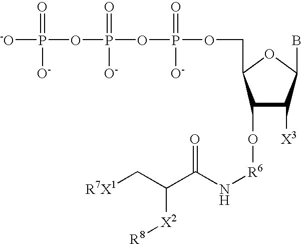

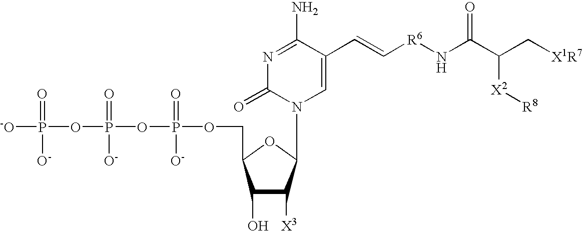

Exemplary nucleotide triphosphates containing a nucleophilic group are set forth in Table 1 above. X¹ and X² represent a first nucleophilic group and a second nucleophilic group, respectively. X³ represents a hydrogen or a hydroxyl. R⁷ and R⁸ individually represent hydrogen or protecting groups. R⁶ represents an appropriate alkylene group as described above. B represents a nucleic acid base.

The nucleotide triphosphates set forth in Table 1 may be produced using any appropriate method. In an exemplary embodiment, a substituted deoxyuridine triphosphate (dUTP) is produced according to Exemplary Scheme 5.

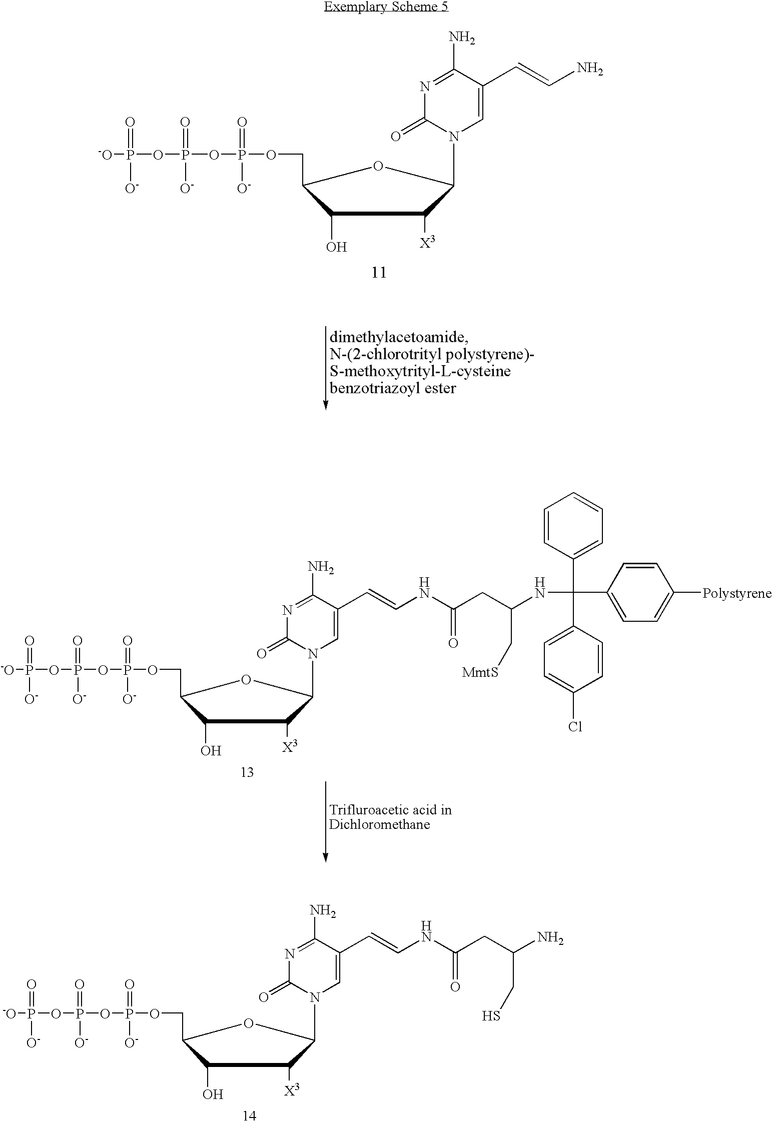

In Exemplary Scheme 5, an aminoallyl dUTP 11 is contacted with a protected solid phase cysteine 12 and dimethylacetamide to yield the solid phase substituted nucleotide 13. Treatment with trifluoroacetic acid provides the corresponding solution phase substituted nucleotide 14.

In another exemplary embodiment, a substituted nucleic is formed by coupling a nucleotide triphosphate containing a reactive group to a nucleic acid. The reactive group is then covalently bonded to a molecule containing at least one nucleophilic group. For example, a double stranded DNA containing a previously incorporated 11 may be reacted with 12 to directly yield a solution phase substituted nucleic acid. In an exemplary embodiment, incorporation is performed enzymatically with a ligase or polymerase. In another exemplary embodiment, incorporation is performed chemically using a phosphoramidite moiety in place of the triphosphate moiety of 11.

The reactive nucleophilic groups of the current invention may be protected or deprotected when enzymatically coupled to the nucleic acid. In an exemplary embodiment, the nucleophilic groups are deprotected before incorporation of enzymatically coupling the nucleotide triphosphate to a nucleic acid molecule. In another exemplary embodiment, the nucleotide triphosphates are enzymatically coupled to the nucleic acid molecule with the nucleophilic groups protected. In another exemplary embodiment, the nucleotide triphosphates are enzymatically coupled to the nucleic acid molecule with at least one nucleophilic groups protected and at least one nucleophilic group deprotected.

In another embodiment, a nucleotide triphosphate containing a nucleophilic group is enzymatically coupled to an internal location within a double stranded nucleic acid sequence. In an exemplary embodiment, one strand of the double stranded nucleic acid is nicked with an endonuclease enzyme. Next, a nucleotide triphosphate containing a nucleophilic group is contacted with polymerase or ligase and the nicked double stranded nucleic acid to form a double stranded substituted nucleic acid wherein the nucleic acid is substituted internally.

In another exemplary embodiment, a substituted phosphoramidite is coupled to a solid phase nucleic acid to form a substituted nucleic acid as described above (see Exemplary Scheme 5). The substituted nucleic acid is then used a primer in a PCR reaction to produce a plurality of modified double stranded nucleic acids. Thus, a single stranded substituted nucleic acid may be lengthened and converted to a double stranded nucleic acid simultaneously.

In another embodiment, a nucleotide triphosphate containing a nucleophilic group is enzymatically coupled to a double stranded nucleic acid containing a first end and a second end. The first and second ends are contacted with a polymerase enzyme and a nucleotide triphosphate containing a reactive nucleophilic group to form a (bis)substituted nucleic acid. The resulting (bis)substituted nucleic acid contains a first end nucleophilic group and a second end nucleophilic group.

In another exemplary embodiment, the first and second ends of the double stranded nucleic acid contain 5′ overhangs. Thus, the nucleotide triphosphate containing a reactive nucleophilic group is added to the recessed 3′ termini of the double stranded nucleic acid. In another exemplary embodiment, the polymerase enzyme is a Klenow polymerase.

In another embodiment, the (bis)substituted nucleic acid is cleaved with an endonuclease to form two substituted double stranded nucleic acids. In an exemplary embodiment, the double stranded nucleic acid is a palendromic sequence containing a endonuclease cleavage site in the middle of the palendromic sequence. Thus, the substituted double stranded nucleic acid products contain double stranded nucleic acids of equal length.

A variety of nucleic acids are useful in forming the substituted nucleic acids of the current invention. In an exemplary embodiment, the nucleic acid is formed using standard solid phase synthesis techniques on a oligonucleotide synthesizer machine. See Beaucage et al., Tetrahedron Lett. 22: 1859 (1981) and Eckstein et al., Oligonucleotides and Analogues: A Practical Approach, (1991). In another exemplary embodiment, the nucleic acid is derived from a cell. For example, cellular DNA or RNA may be isolated from a cell using known techniques and amplified using polymerase chain reaction (PCR) to obtain multiple copies. The resulting nucleic acids may then by substituted with a nucleophilic group, for example, by using the methods presented in the exemplary schemes above.

### II. Methods for Detecting the Presence of A Target Analyte In A Sample

In another aspect, the present invention provides methods of detecting the presence of a target analyte in a sample. The methods include contacting a target analyte with a site specific nucleic acid coupled binding polypeptide to form a target analyte complex, wherein said site specific nucleic acid coupled binding polypeptide comprises a nucleic acid moiety and a binding polypeptide moiety to which the target analyte specifically binds. The nucleic acid moiety of said target analyte complex is contacted with a nucleic acid polymerase to form a plurality of nucleic acid detectors. The nucleic acid detectors are detected thereby detecting the presence of the target analyte in the sample. Typically, the binding polypeptide specifically binds to the target analyte thereby identifying the target analyte.

In one embodiment, the site specific nucleic acid coupled binding polypeptide is formed non-enzymatically according to the methods disclosed above. The use of non-enzymatically formed site specific nucleic acid coupled binding polypeptides provides several advantages. For example, the nucleic acid moiety is coupled to the binding polypeptide via a stable covalent linkage. Thus, the site specific nucleic acid coupled binding polypeptides may be used under a wide variety of conditions to detect target analytes. In addition, the nucleic acids are coupled a specific site on the binding polypeptide with a defined stoichiometry. The defined stoichiometry and location provides accuracy and unambiguousness in interpreting the detection results, especially where detection is by quantitation. Another advantage is that the non-enzymatic methods of making the site specific nucleic acid coupled binding polypeptides are more efficient, higher yielding, and less expensive than the known enzymatic methods.

In another embodiment, the site specific nucleic acid coupled binding polypeptide contains a binding polypeptide moiety covalently bound to a ribonucleic acid moiety. The ribonucleic acid moiety is non-covalently bound to a single stranded deoxyribonucleic acid detector molecule. In an exemplary embodiment, the ribonucleic acid moiety is separated from the deoxyribonucleic acid detector molecule and degraded with a ribonuclease. The single stranded deoxyribonucleic acid detector molecule is contacted with a DNA polymerase to form a double stranded deoxyribonucleic acid detector molecule. The double stranded deoxyribonucleic acid detector molecule is contacted with an RNA polymerase to form a plurality of ribonucleic acid detector molecules. The ribonucleic acid detector molecules are detected thereby detecting said target analyte in the sample. In another exemplary embodiment, the single stranded deoxyribonucleic acid detector molecule is formed by contacting said ribonucleic acid moiety with a reverse transcriptase. In another exemplary embodiment, the RNA polymerase is T7 RNA polymerase and the double stranded deoxyribonucleic acid detector molecule contains a T7 promoter sequence.

In an exemplary embodiment, the detection of the target analyte is accomplished by quantification. Detection by quantification is typically accomplished by quantitating the nucleic acid detectors. Quantitation of nucleic acid detectors may be accomplished by any appropriate technique. Techniques useful in quantitating nucleic acid detectors include, for example, those based on gel electrophoresis (e.g., agarose or polyacrylamide gels), liquid chromatography (e.g. HPLC), and mass spectrometry. Quantitation methods useful in the present invention may be based on a variety of properties, including, for example, fluorescence (see Haugland, Handbook of Fluorescent Probes and Research Chemicals, Molecular Probes, Eugene, (1992)), radioactivity, fluorescence resonance energy transfer (FRET), electrochemilluminescence, chemilluminescence, fluorescence polarization or fluorescence anisotropy, absorbance, and the like. In another exemplary embodiment, a detectable tag is attached to the nucleic acid detector molecule. The detectable tag is typically added to the detector nucleic acid by including a tagged nucleotide that is a polymerase substrate.

In another exemplary embodiment, a fluorescently labeled nucleotide is added during PCR amplification of the nucleic acids moiety to produce a plurality of fluorescently labeled detector nucleic acids. The fluorescently labeled detector nucleic acids are separated from the other PCR reaction components and quantitated based on the fluorescence emission of the fluorescent tag.

In another exemplary embodiment, the nucleic acid polymerase is a heat stable DNA polymerase. Heat stable polymerases of use in the current invention are capable of functioning at elevated temperatures. Typically, the heat stable polymerases are capable of functioning after iterative thermocycles, such as those used in polymerase chain reaction (PCR) techniques (e.g. Taq polymerase). In another exemplary embodiment, the nucleic acid polymerase is an RNA polymerase.

In another exemplary embodiment, the sample comprises at least two different target analytes. The at least two different target analytes are typically detected simultaneously. In an exemplary embodiment, the sample contains two different target analytes. The sample is contacted with two different site specific nucleic acid coupled binding polypeptides. The first site specific nucleic acid coupled binding polypeptide has a nucleic acid moiety that is detectably different than that of the second site specific nucleic acid coupled binding polypeptide.

A variety of properties may provide a detectable difference between the nucleic acid moieties, such as nucleic acid base composition and nucleic acid length. In an exemplary embodiment, the detectable difference is a difference in the length of the respective nucleic acid moieties. The first and second site specific nucleic acid coupled binding polypeptides are contacted with a nucleic acid polymerase to form a plurality of first and second nucleic acid detectors, respectively. The first and second nucleic acid detectors are separated based on the difference in the length between the first and second nucleic acid detectors. Separation may be accomplished using any appropriate technique such as gel electrophoresis, HPLC, capillary electrophoresis and the like. The separated first and second nucleic acid detectors are then detected thereby indicating the presence of two different target analytes. Detection may be accomplished using any appropriate techniques such as those based on absorbance, radioactivity, dye staining, fluorescent labeling, mass (e.g. mass spectrometry) and the like.

In another exemplary embodiment, the detectable difference is the sequence of the nucleic acid moiety. The first and second site specific nucleic acid coupled binding polypeptides are contacted with a nucleic acid polymerase to form a plurality of first and second nucleic acid detectors, respectively. The first and second nucleic acid detectors are separated based on the difference in the sequence between the first and second nucleic acid detectors. A variety of known separation techniques may be employed, such as capillary electrophoresis, affinity chromatography, or microarrays or chips containing affinity reagents (e.g. complimentary single stranded nucleic acids attached to the chip or microarray surface). In addition, the first and second nucleic acid detectors may be detected using mass spectrometry to sequence or partially sequence the nucleic acid detectors either before or after separation.

In another exemplary embodiment, the target analyte is in solution phase. Typically, the solution is an aqueous solution and the target analyte is soluble in aqueous solution. In another exemplary embodiment, the site specific nucleic acid coupled binding polypeptide is contacted with a solution phase target analyte thereby forming a target analyte complex. The target analyte complex is then isolated from the solution mixture using known separation techniques, such as gel electrophoresis, column chromatography and the like. The isolated target complex is contacted with a nucleic acid polymerase to form the nucleic acid detector molecules.

In another exemplary embodiment, the method also includes immobilizing a target analyte onto a solid support. The target analyte is typically immobilized before contacting the target analyte with a site specific nucleic acid coupled binding polypeptide. A variety of solid supports are useful in the present invention, including, for example, beads (including magnetic beads), resins, biochips and the like. Solid supports may contain a variety of materials. Support materials are typically chosen so as not to disrupt the reactions of the method. Useful solid support materials include, for example, agarose, polyacrylamide, controlled pore glass, PMMA, cellulose, latex, optionally functionalized polystyrene, optionally substituted copolymers of polyethylene glycol (PEG)-polystyrene (PS) (see Castelhano et al., U.S. Pat. No. 6,376,667 which is herein incorporated by reference for all purposes), Tentagel™ beads (Ohlmeyer et al., Proc Natl Acad Sci 90:10922-10926 (1993), glass, Wang resin, Rapp resin, silica gels, glass particles coated with hydrophobic polymer, etc., i.e., material having a rigid or semi-rigid surface, soluble supports such as low molecular weight non-cross-linked polystyrene, plasma-modified plastics (e.g. plasma-modified polypropylene and other plasma-modified plastics used in PCR). Other support materials that are known in the art and can be used without departure from the scope of the present invention are also included, such as those described in Jung et al., Combinatorial Peptide and Nonpeptide Libraries, A Handbook (1996) or Bunin et al., The Combinatorial Index (1998) which are incorporated herein by reference.

In another exemplary embodiment, the at least two different target analytes are bound to a solid support. In another exemplary embodiment, the solid support is a magnetic solid support covalently bound to at least two different target analytes. Typically, the magnetic solid support is beadlike or spherical in shape.

In another exemplary embodiment, at least two different site specific nucleic acid tagged binding polypeptides are added to a sample containing a target analyte. Each of the at least two different site specific nucleic acid tagged binding polypeptides are capable of binding to the target analyte. By using at least two different site specific nucleic acid tagged binding polypeptides that bind to the same target analyte, identification and quantitative accuracy are increased.

Any appropriate target analyte may be detected using the methods of the present invention. In an exemplary embodiment, the target analyte is a chemical or biochemical analyte. In another exemplary embodiment, the target analyte is a protein, a carbohydrate, a nucleic acid, a lipid, a vitamin, a virus, a bacteria, or an inorganic molecule.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, any feature of the methods of forming a site specific nucleic acid coupled binding polypeptide detecting can be incorporated into any of the methods of detecting a target analyte without departing from the scope of the invention.

In addition, the patents and scientific references cited herein are incorporated by reference in their entirety.

### EXAMPLES

### Example 1

Example 1 illustrates an exemplary method of introducing into a cell a recombinant DNA molecule comprising a nucleotide sequence encoding a recombinant binding polypeptide-intein molecule, wherein the recombinant binding polypeptide-intein molecule comprises an intein moiety genetically engineered into a predetermined site on a binding polypeptide moiety.

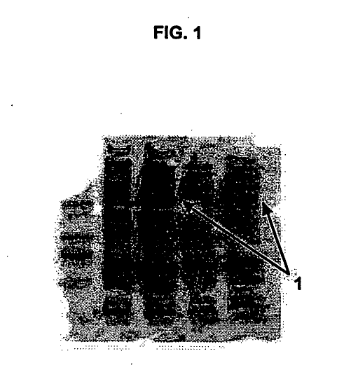

A gene encoding a binding polypeptide was cloned in frame to an amino terminal end of a Saccharomyces cerevisiae VMA intein coding sequence. The binding polypeptide-intein nucleic acid sequence was inserted into the bacterial expression vector pTYB1, which is under the control of an inducible promoter. The vector was then transformed into Escherichia coli ER2256. The bacterial cells were grown to an Optical Density (OD) of 0.8 at 600 nm, and induced using 1 mM isopropyl thiogalactopyranoside (IPTG) for four hours. Bacterial cells were collected by centrifugation, lysed and subjected to 12% acrylamide gel electrophoresis followed by staining the Coomassie blue. A photograph of a gel with 5 lanes is presented in FIG. 1.

Lane 1 (far left) is a protein size standard. Lanes 2 and 3, represent the binding polypeptide-intein molecule wherein the binding polypeptide is a polypeptide aptamer that binds human cyclin-dependent kinase 2 (hCDK2). Lane 3 depicts the cell lysate after induction of the polypeptide aptamer-intein encoding sequence and Lane 2 depicts the un-induced polypeptide aptamer-intein sequence. As shown in Lane 3, induction of the polypeptide aptamer-intein sequence produces the polypeptide aptamer-intein fusion protein at the expected size at arrow 1.

Lanes 4 and 5 represent the products of an uninduced streptavidin-intein sequence and an induced streptavidin-intein sequence, respectively. Again, the streptavidin-intein molecule product is shown at the expected size in Lane 5 by arrow 1.

### Example 2

Example 2 illustrates an exemplary method of forming a substituted double stranded nucleic acid.

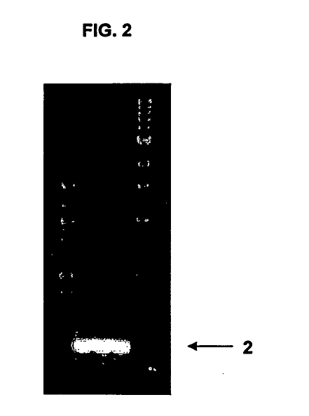

A substituted phosphoramidite containing a sulfhydryl first nucleophilic group protected with S-tert-butyl sulfonyl and an amino second nucleophilic groups protected with Fmoc was coupled to a solid phase nucleic acid molecule using standard phosphoramidite coupling conditions. Treatment with ammonium hydroxide resulted in Fmoc deprotection and cleavage from the solid support, thus yielding the di-substituted nucleic acid molecule. The di-substituted nucleic acid molecule was used as a PCR primer to produce the corresponding di-substituted double stranded nucleic acid. The di-substituted double stranded nucleic acid was isolated by gel electrophoresis as shown in FIG. 2. Arrow 2 indicates the isolated di-substituted double stranded nucleic acid in lanes 2-4. Lane 1 (far left and lane 5 (far right) contain control marker nucleic acids.

### Example 3

Example 3 illustrates an exemplary method of forming a site specific nucleic acid coupled binding polypeptide using a recombinant polypeptide-intein molecule.

The streptavidin-intein molecule of Example 1 was contacted with thiophenol to yield the streptavidin-thiophenol intermediate. The isolated di-substituted double stranded nucleic acid of Example 2 was contacted with the streptavidin-thiophenol intermediate in the presence of tris(2-carboxyethyl)phosphine (TCEP) to yield the site specific di-substituted nucleic acid coupled streptavidin. The site specific di-substituted nucleic acid coupled streptavidin was purified by ion-exchange HPLC followed by reverse phase HPLC.

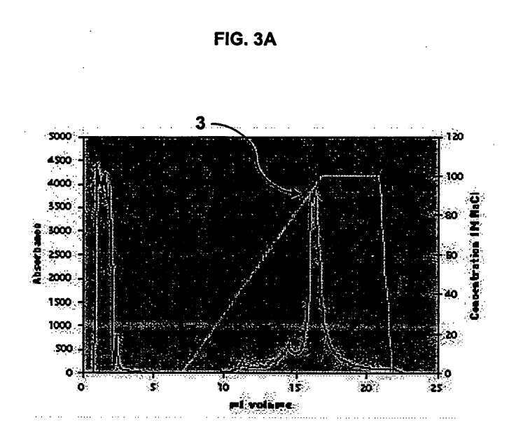

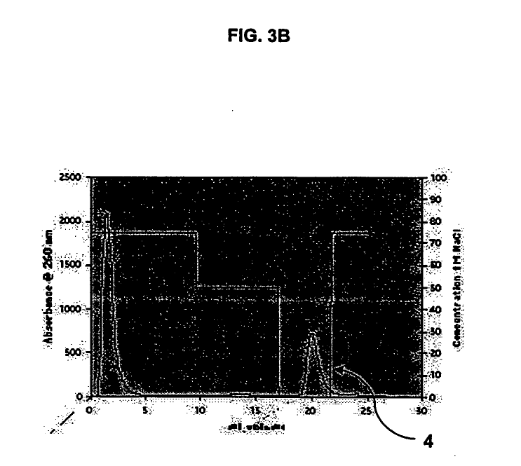

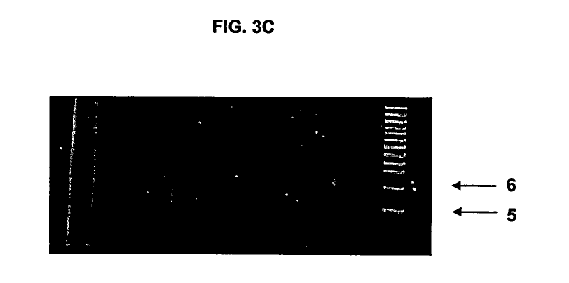

FIG. 3A-3C show the purification and detection of the site specific di-substituted nucleic acid coupled streptavidin product. FIG. 3A is a chromatograph of an ion-exchange HPLC. Arrow 3 indicates the major peak containing the site specific di-substituted nucleic acid coupled streptavidin and unreacted di-substituted double stranded nucleic acid. FIG. 3B is a chromatograph of a reverse phase HPLC. Arrow 4 indicates the major peak containing the purified site specific di-substituted nucleic acid coupled streptavidin.

FIG. 3C is a photograph of reverse phase HPLC fractions after gel electrophoresis and treatment with ethidium bromide. Arrows 5 and 6 represent the unreacted di-substituted double stranded nucleic acid and the site specific di-substituted nucleic acid coupled streptavidin, respectively. Lane 1 (far left) is the unpurified reaction mixture. Lane 2 is the ion-exchange peak indicated by arrow 3. Lanes 4 and 9 are di-substituted double stranded nucleic acid standards. Lane 10 (far right) contains control marker nucleic acids. Lanes 6-8 contain the fractions from the reverse phase HPLC peak indicated by Arrow 4.

### Example 4

Example 4 illustrates an exemplary method of contacting a target analyte with a site specific substituted nucleic acid coupled polypeptide to form a target analyte complex.

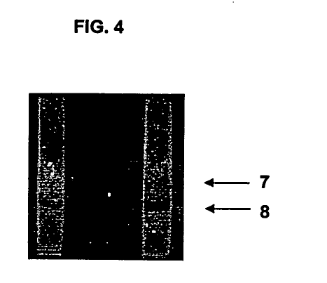

The purified site specific di-substituted nucleic acid coupled streptavidin of Example 3 was added to a solution containing biotin-labeled bovine serum albumin (BSA) resulting in a complex between the streptavidin moiety and the biotin moiety. The complex was detected by gel electrophoresis, as shown in FIG. 4.

FIG. 4 is a digital representation of the complex formation after gel electrophoresis and treatment with ethidium bromide. The complex is indicated by arrow 7 and the unbound site specific di-substituted nucleic acid coupled streptavidin is indicated by arrow 8. Lane 1 (far left) is the purified site specific di-substituted nucleic acid coupled streptavidin. Lane 2 is the biotin BSA without the addition of site specific di-substituted nucleic acid coupled streptavidin. Lane 3 (far right) is the site specific di-substituted nucleic acid coupled streptavidin and biotin BSA reaction mix.

What is claimed is:

1. A method of forming a site specific nucleic acid coupled binding polypeptide comprising:
(i) introducing into a cell a recombinant DNA molecule comprising a nucleotide sequence encoding a recombinant binding polypeptide-intein molecule, wherein the recombinant binding polypeptide-intein molecule comprises an intein moiety genetically engineered into a predetermined site on a binding polypeptide moiety;
(ii) isolating the recombinant binding polypeptide-intein molecule from the cell;
(iii) contacting the recombinant binding polypeptide-intein molecule with a nucleophile under conditions which permit the substitution of the intein moiety with the nucleophile to yield a binding polypeptide intermediate comprising a binding polypeptide moiety and a nucleophile moiety covalently linked through a first bond; and
(iv) contacting the first bond with a substituted nucleic acid under conditions which permit substitution of the nucleophile moiety with the substituted nucleic acid to form a site specific nucleic acid coupled binding polypeptide comprising a binding polypeptide moiety and a substituted nucleic acid moiety covalently linked through a second bond, wherein said substituted nucleic acid comprises a first nucleophilic group.

2. The method of claim 1, wherein
said nucleophile is selected from the group consisting of thiol and alcohol;
said first nucleophilic group is selected from the group consisting of sulfhydryl and hydroxyl; and
said first bond and second bond are independently is selected from the group consisting of thioester and an ester;.

3. The method of claim 1, wherein said substituted nucleic acid comprises a recombinant nucleic acid moiety.

4. The method of claim 1, wherein said substituted nucleic acid further comprises a second nucleophilic group, wherein said second nucleophilic group displaces the first nucleophilic group to form a third bond between the binding polypeptide moiety and the substituted nucleic acid moiety.

5. The method of claim 4, wherein
said second nucleophilic group is selected from the group consisting of hydroxyl and amino;
said third bond is selected from the group consisting of ester and amide.

6. The method of claim 1, wherein said nucleophile is HS—R, wherein R is selected from the group consisting of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

7. The method of claim 1, wherein said nucleophile is selected from the group consisting of thiophenol and 2-mercaptoethanesulfonic acid.

8. The method of claim 1, further comprising, before step (iv), the step of forming a substituted nucleic acid, wherein said step comprises contacting a substituted nucleotide with a polymerase and a double stranded nucleic acid to form a double stranded substituted nucleic acid.

9. The method of claim 8, wherein said double stranded nucleic acid comprises a first end and a second end, wherein:
(a) said first and said second end are contacted with an enzyme and a substituted nucleotide to form a (bis)substituted nucleic acid comprising a first end nucleophilic group and a second end nucleophilic group; and
(b) contacting said (bis) substituted nucleic acid with a nuclease to form at least two double stranded substituted nucleic acids.

10. The method of claim 8, wherein said enzyme is a member of the group selected from a polymerase and a ligase.

11. The method of claim 1, further comprising, before step (iv), the step of forming the substituted nucleic acid, wherein said step comprises contacting a substituted phosphoramidite with a nucleic acid to form a substituted nucleic acid.

12. The method of claim 1, wherein said binding polypeptide is selected from the group consisting of an antibody, a hormone, and a cell-surface receptor.

13. A method for detecting the presence of a target analyte in a sample comprising:
(i) contacting a target analyte with a site specific nucleic acid coupled binding polypeptide to form a target analyte complex, wherein said site specific nucleic acid coupled binding polypeptide comprises a nucleic acid moiety and a binding polypeptide moiety to which the target analyte specifically binds, wherein said site specific nucleic acid coupled binding polypeptide is formed non-enzymatically;
(ii) contacting the nucleic acid moiety of said target analyte complex with a nucleic acid polymerase to form a plurality of nucleic acid detectors;
(iii) detecting said nucleic acid detectors thereby detecting the presence of the said target analyte in the sample.

14. The method of claim 13, wherein said detecting is by quantification.

15. The method of claim 13, wherein said nucleic acid polymerase is a heat stable DNA polymerase.

16. The method of claim 13, wherein said nucleic acid polymerase is an RNA polymerase.

17. The method of claim 13, wherein said sample comprises at least two different target analytes.

18. The method of claim 13, further comprising, before step (i), immobilizing the target analyte onto a solid support.

19. The method of claim 13, wherein said target analyte is in solution phase.

20. The method of claim 13, wherein said target analyte is selected from the group consisting of a protein, a carbohydrate, a nucleic acid, a lipid, a vitamin, a virus, a bacteria, and an inorganic molecule.

21. A method for detecting a target analyte in a sample comprising:
(i) contacting a target analyte with a site specific nucleic acid coupled binding polypeptide to form a target analyte complex, wherein said site specific nucleic acid coupled binding polypeptide comprises a binding polypeptide moiety covalently bound to a ribonucleic acid moiety, wherein said ribonucleic acid moiety is non-covalently bound to a single stranded deoxyribonucleic acid detector molecule;
(ii) separating the ribonucleic acid moiety from the deoxyribonucleic acid detector molecule;
(iii) degrading the ribonucleic acid moiety with a ribonuclease;
(iv) contacting the single stranded deoxyribonucleic acid detector molecule with a DNA polymerase to form a double stranded deoxyribonucleic acid detector molecule;
(v) contacting the double stranded deoxyribonucleic acid detector molecule with an RNA polymerase to form a plurality of ribonucleic acid detector molecules;
(vi) detecting said ribonucleic acid detector molecules thereby detecting said target analyte in the sample.

22. The method of claim 21, wherein said detecting is by quantification.

23. The method of claim 21, wherein said target analyte is a member selected from the group of a protein, a carbohydrate, a nucleic acid, a virus, a bacteria, and an inorganic molecule.

24. The method of claim 21, wherein said sample comprises at least two different target analytes.

25. The method of claim 21, further comprising, before step (i), immobilizing the target analyte onto a solid support.

26. The method of claim 21, wherein said target analyte is in solution phase.

27. The method of claim 21, wherein said single stranded deoxyribonucleic acid detector molecule is formed by contacting said ribonucleic acid moiety with a reverse transcriptase.

28. The method of claim 21, wherein:
said RNA polymerase is T7 RNA polymerase; and
said double stranded deoxyribonucleic acid detector molecule comprises a T7 promoter sequence.
